(19) United States
(12) Patent Application Publication
(10) Pub. No.: US 2014/0100620 A1
(43) Pub. Date: Apr. 10, 2014

(54) Laser Projected Display for Implant Orientation and Placement

(71) Applicant: Vector Sight Inc., Germantown, TN (US)

(72) Inventor: Michael W. Mullaney, Naples, FL (US)

(21) Appl. No.: 14/044,382

(22) Filed: Oct. 2, 2013

Related U.S. Application Data
(60) Provisional application No. 61/793,645, filed on Mar. 15, 2013.
(60) Provisional application No. 61/708,820, filed on Oct. 2, 2012.

(51) Int. Cl. A61F 2/46; A61B 17/88

(52) U.S. Cl. CPC A61F 2/46; A61B 17/88
USPC 606/86 R

(57) ABSTRACT

A Laser projection system for identifying a point in space includes a Laser projector capable of generating more than one beam of light. A system is configured to define the location of a point or collection of points either through the intersection of the beams of light in three-dimensional space or the intersections of the beams of light with a known surface topology in order to define a position and pose in three dimensional space.

### PRIORITY

This disclosure claims priority to and the benefit of the filing date of U.S. provisional patent application 61/708,820, filed Oct. 2, 2012 and U.S. provisional patent application 61/793,645, filed Mar. 15, 2013, both of which are incorporated herein by reference.

### FIELD OF THE INVENTION

This disclosure relates to systems that aid in the location of a particular implant or element of an implant that is disposed within a patient.

### BACKGROUND

Modern medicine has come to provide many solutions to many common medical problems in the way of devices that are implanted within the body. Hip replacements, knee replacements, plates, screws, and nails for trauma related injuries etc. find their way into a large percentage of the human population over the course of a typical lifetime. In each case these devices need to be implanted with a high degree of placement accuracy if the device is to perform as desired. Achieving such accuracy is often difficult because in most cases body tissues obscure the implant making visualization in situ a challenging task. Such conditions often result in subpar performance and in many instances lead to a repeat procedure, which is both costly to the quality of life of the individual and medical field at large.

One such application that would benefit from a system for achieving accuracy in implant placement is in the field of intramedullary nailing, specifically the targeting of the interlocking screws. These are amongst the most challenging implants to place owing to the fact that the features being targeted are completely obscured by intact bone and can only be seen with the use of intraoperative X-rays which are dangerous to both the patient and to the surgical staff. Surgeons typically use either several x-ray pictures or live fluoroscopy to freehand the placement of these interlocking screws or they resort to mechanical fixtures in conjunction with other methods to achieve proper alignment.

Many other such examples exist related to other types of implants where the same concerns apply. In each case, placement, whether it is alignment along a single axis such as was the case in the interlocking screw example or alignment with a multitude of axes as would be the case in the placement of a knee replacement component the relationship between the implant and the patient anatomy must be correctly controlled and perhaps more importantly that relationship must be adequately and easily communicated to the operative surgeon and support staff.

The systems and methods disclosed herein overcome one or more of the shortcomings in the industry.

### SUMMARY

This disclosure provides systems and methods for annotating on one or both of the patient's body and on an implant or instrument itself real time intuitive positional information that the surgeon can utilize without them having to remove their attention from either the instrument or the patient in front of them. In other words, a heads up sort of display where the graphical information is “projected” directly on both the patients anatomy and the instrument or implant that is to be guided. These systems aid in identifying the location of a particular implant or element of an implant that is disposed within a patient. Specifically it refers to the virtual display of such geometric characteristics that can be denoted with a collection of lines displayed in space that are constructed by the intersection of sheets of light and a surface. The surface can include, but is not limited to, a patient's anatomy, part of an instrument or part of the implant itself.

In an exemplary aspect, the present disclosure is directed to a surgical system or jig that displays graphical data to target any desired axial trajectory which in this case would correspond to one of the cross-locking screw holes in the nail itself. It should be further noted that this same system could be expanded to target multiple axes simultaneously if the need arose but for the sake of simplicity and clarity this disclosure will focus on the method for targeting a single axis.

The jig or system includes an attachment mechanism affixed to an implant or instrument in question or the patient anatomy in a position relative to the implant or instrument. In some alternative aspects, the jig is placed remote to the patient and/or implant/instrument and its location is registered with respect to the patient or implant using known systems such as a computer assisted surgery system (CAS). The attachment mechanism or the registered location would serve as a reference base for the attachment of one or more extensible structures that include a movable device having at least two degrees of freedom and the capability to direct the trajectory of a light source. An example of such a light source can be found in Laser galvo minor scanners which utilize either rotating or vibrating minors to direct a beam of light. Other methods exist that use optical approaches such as the use of cylindrical lenses as described in U.S. Pat. No. 5,095,386 to Scheibengraber, Optical System for Generating Planes of Light Using Crossed Cylindrical Lenses. Still other optical systems exist to create a variety of open and closed curves of light. A main difference between these systems is that, in some instances, the moving devices redirect light that is incident upon them utilizing minors whereas in others the moving devices move and direct the light source. The first configuration is the one that is most often used as it is much easier to redirect a reflected beam of light than it is to redirect a light source owing to inertial effects.

These beams of light may be redirected at a high frequency using the movable platforms such that they target a continuous set of arbitrary points in space. These arbitrary points in space usually will take the form of a curve with each successive point being in close proximity to the previous and typically these points will be targeted repeatedly, the effect being that any arbitrary curve can be painted on any arbitrary surface. In the simplest of cases, this arbitrary curve is a straight line and the arbitrary surface is a plane but other open or closed curves could be drawn on surfaces including but not limited to flat planes.

It is common practice in a number of fields to use Lasers to help in positioning. An example of this is the use of a Laser level to define a position along a wall. A Laser level is the combination of a Laser that projects a plane and a bubble level. The user positions the level so that the bubble is in the orientation desired. The Laser projects a plane, allowing the user to visualize the intersection of the object of interest with the projected plane, for example the intersection of the wall with the Laser projected plane for placing fasteners. The prior art devices therefore do not supply the user with a unique position solution in unbounded or arbitrary 3d space, but provide the user with a bounded region of space defined by the projection of the Laser, and the user decides how to use that bounded region. This works well when there are other limits implied by the use of the device. For example, when using a Laser sight to orient a mobile fluoroscope, the user is interested in aligning the fluoroscope with the anatomy of interest. Since the Laser sight on the fluoroscope is fixed relative to the X-ray beam, placing the Laser light over the anatomy of interest places the X-ray beam over the anatomy of interest. This Laser sight does not provide the user a unique solution in unbounded or arbitrary 3d space for placement of the fluoroscope, so the user uses other inputs to decide the final position and pose. This can be adequate for devices with limited degrees of freedom, such as a drill press, or for devices where the user can visualize the proper placement using other methods of alignment. It is inadequate in the cases where you cannot see what you are aiming at, such as distal targeting of an intramedullary nail, or where additional degrees of freedom cannot easily be determined, such as establishing a desired orientation of a joint replacement relative to patient anatomy. The systems and methods disclosed herein differs from the above described systems in that it provides the user with a visual display of a unique positional solution in unbounded or arbitrary 3d space, whether that unique solution is a single point, an axis or trajectory, or a plane or 3D coordinate system.

With regard to surgical techniques requiring the proper placement of either an implant or instrument in relation to the body the simplest of cases is one where a trajectory must be identified. To identify such a trajectory one must define an axis which can simply be a straight line drawn between two plotted points on a plane that the axis lies within or the intersection of two planes in 3d space. Since the medical workspace seldom comes with predefined accurately positioned work planes onto which points can be drawn and axes plotted between, having the capability to create and utilize two intersecting planes in 3d space is a much more powerful solution.

If it is desired to not only identify a trajectory but also to denote points along that trajectory then the aforementioned lines denoting the axis simply need to be initiated, then interrupted or ended at the point of interest. Alternatively, the plotted lines depicting the axis having been created by the intersection of two planes could be intersected by a curve drawn on either of those planes or a third surface the axis intersects. If it is desired to identify a trajectory, a point on that trajectory and a rotation about that trajectory, i.e. a coordinate system, then one must also define a second axis utilizing a second pair of intersecting planes. This second axis would intersect the first and be at a known angular orientation, preferably orthogonal. The two intersecting axes would define a plane. A normal to that plane from the point of intersection of the first two axes would provide the third orthogonal axis, i.e. a coordinate system, which is all that is needed to specify the position and pose of any object in 3d space.

In order to accurately target the cross locking holes in an intramedullary nail the location and orientation of the cross locking holes geometry must be known with respect to the jig or targeting device. Since the geometry of the nail in the free state is known through product dimensions and subsequent calibration steps and since the deformed state due to implantation is also known through measurement or other means that exist in the field it is a simple matter to selects two points in 3d space that lie on the axis of interest. These first two points are then combined with a point corresponding to the first light source on the targeting jig to create a first plane and then the same two points or others lying on the axis of interest are then combined with the point corresponding to the second light source to form a second plane, the point of the first light source and the point of the second light source being separate in 3d space. Each light source is then commanded to target a sequence of closely spaced points along the axis of interest between the two first identified points on the axis of interest in rapid and repeating succession each thereby creating a plane of light that intersects the axis of interest and which are non-coincident. The benefit of the lack of coincidence of these two planes of light generated by the disparate sources of light will be made apparent once the planes of light are cast upon the patient

When these planes of light are cast upon the patient anatomy and any article of interest, such as a drill guide for example, they form lines on the intersected surface of the patient anatomy and the surface of the drill guide. Each plane of light forms its own line, and the intersection of these lines on the patient may also be coincident with the desired axial trajectory. The surgeon simply creates a stab wound at the intersection of these lines, inserts the drill guide until it is firmly against the bone, and pivots the drill guide about the point of contact with the bone until lines formed by the sheets of light projected onto the drill guide converge into a single line on planar surfaces within which the axis lies. Where the surface of the drill guide is oblique to the axis of interest then one must adjust the drill guide until the lines intersect at a point on the surface that is coincident to the axis of interest. These lines could have their beginning or ending controlled or be interrupted along their length at desired location to denote a location along the lines. Similarly a third sheet of light may be cast upon the drill guide to create an intersection with the desired axis, that intersection being a point of reference along that desired trajectory. Either of these methods may be utilized for things such as depth of insertion of a hip stem or the axis and depth of a hip socket reamer. In some aspects, a second pair of intersecting sheets of light may be combined with the first pair to denote a second axis of interest and so on leading to fully described coordinate system. With this approach, a surgeon can easily appreciate that any implant of interest can be fully described in terms of both position and pose in relation to a particular patients anatomy and/or in relation to itself in some known (un-deflected in the case of an intramedullary nail (“IMN”)) condition.

In summary for the simple example of targeting cross locking holes in an IM nail the first two intersecting planes denote the axis of interest, crossing this axis with a third plane denotes a point along that axis and the display of a second axes in relation to the first through the use of two additional planes would describe a rotation about the first axis. Since the movable platforms from which the sheets of light emanate are designed to have a sufficient mobility, the axes can be generated as freely and as precisely as is required. Such ability can easily be adapted to other such applications where position and pose are important, such as hip stem or cup preparation and insertion, knee replacement preparation and insertion, and others.

Similar methods may be used to denote placements in 3d space that only utilize a single light source having multiple degrees of freedom. Since the light can be directed along any vector having an origin at the source and having a trajectory lying within the working envelope that describes the mobility of the moving platform or minor, etc. a single light source can project an arbitrary shape onto any surface that intersects the working envelope of the light source. In one aspect such a shape could be a rectangle with the arbitrary surface being a plane at a known position in space. If an instrument or implant were to have a planar surface with an inscribed or similarly marked rectangle one could utilize such a light projection system to accurately position and pose that instrument or implant in space by making certain that the inscribed rectangle on the instrument or implant was illuminated by the rectangle projected by the light source.

In an exemplary aspect, the present disclosure is directed to a Laser projection system for identifying a point in space. The system includes a first Laser projector configured to generate a first beam at a first angle and includes a second Laser projector configured to generate a second beam that intersects the first beam at an intersection. A system is configured to determine a location of a point of the intersection of the first and second beams in three-dimensional space.

In an aspect, the first and second Laser projectors are each configured to generate beams as planar sheets, the intersection of the sheets forming an axis, the system being configured to determine a location of the axis. In an aspect, the system includes a surgical drilling instrument, and wherein the axis is aligned with an outer surface of the drilling instrument for drilling to the feature of an implant in a patient.

In an exemplary aspect, the present disclosure is directed to a Laser projection system for marking an axial target as a part of a surgical implant procedure. The system includes a first Laser projector configured to project a first plane of light and includes a second Laser projector configured to project a second plane of light that intersects the first plane of light. A system is configured to manipulate the Laser projectors so that a line defined by the intersection is aligned to represent the axial target as one of a location of a feature of an implant implanted within a patient and a desired location of an instrument used for implantation of an implant.

In an aspect, the line is aligned with an outer surface of an instrument for drilling to the feature of the implant. In an aspect, each of the first and second Laser projectors comprise a mirror that is vibrated to create the respective first and second planes of light.

In an exemplary aspect, the present disclosure is directed to a Laser projection system for marking a positional target as part of a surgical implant or instrument. The system includes a Laser projector configured to project a visual indicator onto a virtual surface in space at a location on the virtual surface coinciding with the desired placement of a surgical implant or instrument. The system also includes a surgical implant or instrument comprising an outer surface such that when the surgical implant or instrument is placed in a field of view of the Laser projector, the projected visual indicator aligns on the outer surface when the implant or instrument is properly placed in space.

In an aspect, the visual indicator is a line. In an aspect, the system includes a second Laser projector configured to project a second visual indicator intersecting the first visual indicator to form an intersection representing an axis coinciding with the desired placement of a surgical implant or instrument. In an aspect, the Laser projectors are configured to project the indicators on patient anatomy to identify a point coincident with an axis of interest.

In an exemplary aspect, the present disclosure is directed to a Laser projection system for marking an axial target as a part of a surgical implant procedure performed by a surgeon. The system includes an implant feature locator configured to determine the location of a feature of an implant in a patient and determine a target axis to the feature of the implant. The system also includes a first Laser generating system configured to generate a Laser sheet passing through the target axis for the feature of the implant. The system also includes a second Laser generating system offset from the first Laser generating system configured to generate a Laser sheet passing through the target axis for the feature of the implant, the second Laser sheet intersecting the first Laser sheet at the target axis in a manner visible to the surgeon.

In an aspect, the implant feature locator comprises a processing system configured to receive and process dimensional data of the implant to determine the location of the feature of the implant. In an aspect, the processing system is configured to receive data from a deflection probe associated with the implant and configured to calculate deformation of the implant and determine the desired location of the target axis. In an aspect, the target axis is an axis configured to align with an outer surface of an instrument for drilling to the feature of the implant. In an aspect, each of the first and second Laser generating systems comprises a mirror that is vibrated to create the Laser sheet. In an aspect, the first Laser generating system comprises a cylindrical lens that is rotated to create the Laser sheet. In an aspect, the first Laser generating system comprises a mirror disposed on a MEMS chip that moves the mirror in two planes to direct a Laser and form the first Laser sheet. In an aspect, the implant is one of an intramedullary nail, a hip replacement, and a knee replacement.

In an exemplary aspect, the present disclosure is directed to a method for marking an axial target as a part of a surgical implant procedure. The system includes implanting an implant in a patient, the implant comprising a feature of interest; and using a first Laser beam and a second Laser beam that intersect to create a visual point on patient's anatomy identifying a target to the feature of interest of the implant.

In an aspect, the method includes generating the first Laser sheet and a second Laser sheet that intersect create a visual intersecting axis, the visual point being along the intersecting axis, the intersecting axis identifying a target axis to the feature of interest of the implant. In an aspect, the method includes maintaining a surgical instrument in a position where the visual intersecting axis forms a line on the instrument to provide an indicator of whether the surgical instrument is aligned with the target axis to the feature of interest of the implant.

In an exemplary aspect, the present disclosure is directed to a method of determining the location of a surgical implant or instrument, the method including implanting a reference implant; determining the location of the reference implant relative to a Laser system; illuminating one or more points identifying a location relative to the reference implant with the Laser system; and aligning instruments or implants utilizing the one or more illuminated points.

In an aspect, the Laser system comprises two spatially separate Laser sources and wherein illuminating one or more points with the Laser system comprises orienting Lasers from the two Laser sources so that they intersect to illuminate the one or more points with the Laser system. In an aspect, illumination of one or more points comprises: illuminating items placed in a section of a first plane generated by the first Laser source; illuminating items placed in a section of a second plane generated by the second Laser source, wherein the intersection of the first plane and the second plane defines an axis of interest; and aligning instruments or implants comprises utilizing the axis of interest. In an aspect, the method includes illuminating items placed in a section of a third plane generated by the first Laser source; and aligning the implant or instrument using the third plane. In an aspect, the method includes illuminating items placed in a section of a fourth plane generated by the second Laser source, wherein the intersection of the third plane and the fourth plane defines a second axis of interest; and aligning instruments or implants comprises utilizing the first axis of interest and second axis of interest. In an aspect, the section of the first plane and the section of the second plane end at a desired point of interest along the axis of interest, and wherein aligning instruments or implants comprises aligning along the axis of interest to a position relative to the point of interest. In an aspect, the method includes projecting the one or more illuminated points with a single Laser.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is an illustration of an exemplary Laser projection system in use on a leg of a patient in accordance with one aspect of the present disclosure.

FIG. 2 is an illustration of an exemplary implant usable with the Laser projection system of FIG. 1.

FIGS. 3-4 are illustrations of the Laser projection system in accordance with one exemplary aspect of the present disclosure.

FIG. 5 is an illustration of an exemplary optical system forming a part of the Laser projection system in accordance with one aspect of the present disclosure.

FIG. 6 is an illustration of an exemplary MEMS minor forming a part of the Laser projection system in accordance with one aspect of the present disclosure.

FIG. 7 is an illustration of an exemplary Laser projection system projecting plane sections that define a point and an axis of interest.

FIG. 8 is an illustration of an exemplary Laser projection system projecting plane sections that define two axes of interest and an entire three-dimensional coordinate system.

FIGS. 9A and 9B illustrate an example of a single light source projecting a polygon onto a surface of a virtual plane for object placement in accordance with one aspect of the present disclosure.

FIG. 10 is an illustration of an exemplary instrument with a target polygon, along with a single light source projecting a polygon.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The exemplary Laser projection systems disclosed herein are arranged to direct the placement of an implant, such as intramedullary nails, hip stem and cup implants, knee replacement implants, and others. This may include an axial trajectory identifying the location of screw holes or may include presenting an axial trajectory identifying other features of the implant for anchoring or for general implantation or more generally it could be one or more axial identifiers that correspond to such things as a coordinate system. One system described herein is used to display the position of interlocking screw holes in an IMN in a patient. The system generates a Laser marker that shows a surgeon where to drill and at what angle to drill to engage the interlocking screw hole in the IMN. It should be noted that is merely a single axis application and its description herein is chosen for the sake of simplicity and no such limitation is anticipated or required. It is further anticipated that this single axis example would be expanded to include a full coordinate system definition through the use of multiple axes each defined in similar ways.

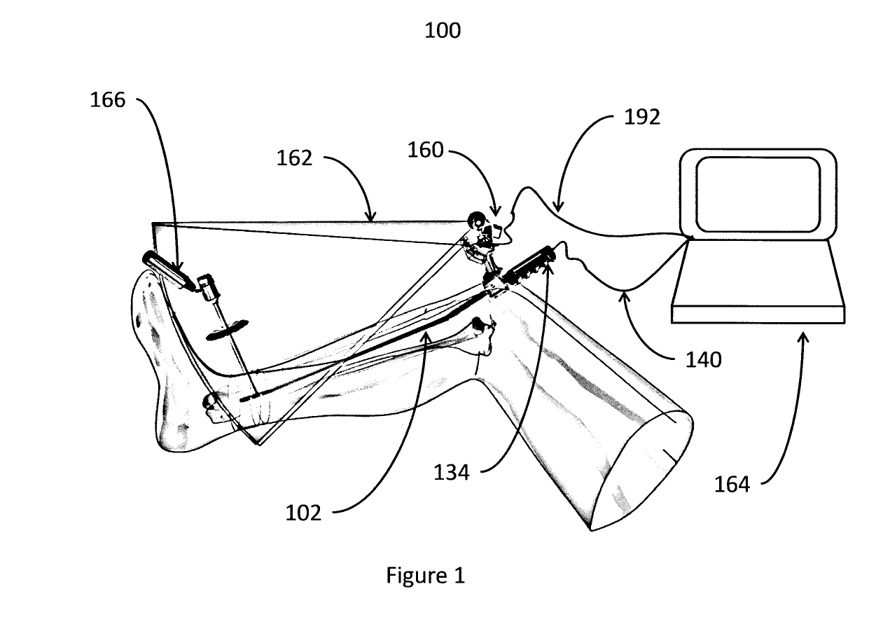

FIG. 1 shows an exemplary Laser projection system 100 in accordance with an exemplary aspect of the present disclosure. The system in FIG. 1 is shown connected to an intramedullary nail, referred to herein as implant 102. While shown as an IMN, the implant 102 could be any implant where axial targeting may be useful whether it be one, two or more axial trajectories that need be identified. The implant 102 may also be a temporary implant placed in or on the bone as a reference marker so that the relationship between the bone and the Laser system 100 can be defined. In the example described, the IMN is the implant 102, and the features formed on the implant 102 that are not visible to a surgeon are interlock holes configured to receive an interlock screw. The Laser projection system 100 may be used to guide an instrument, such as a surgical drill, and may be used to guide an additional connecting implant, such as an interlock screw, into the interlock holes in the intramedullary nail implant 102 when the intramedullary nail is disposed within a patient.

The Laser projection system 100 includes a Laser support structure 160, a single or plurality of Laser projectors 162, and a processing system 164. In some aspects, the Laser projection system 100 may be considered to include a surgical instrument or tool 166.

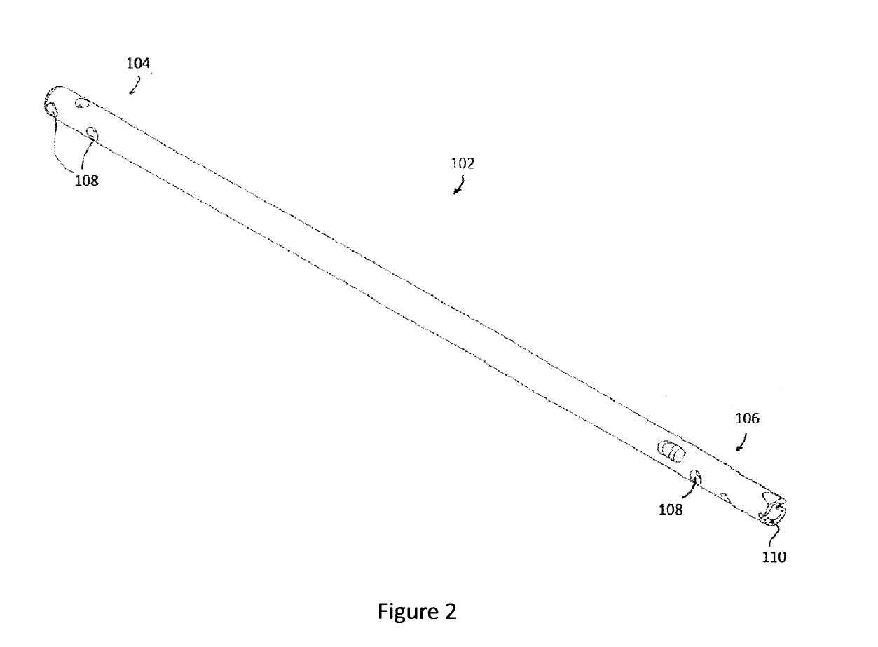

FIG. 2 shows an exemplary implant 102 as an IMN that may be used with the Laser projection system 100. The IMN implant 102 includes a distal end 104, a proximal end 106, and includes interlock holes 108 arranged to receive the interlocking screws (not shown). When the IMN is in the intramedullary canal of the patient, the interlocking screws are driven into the bone and the IMN to prevent relative rotation. In this embodiment, the nail implant 102 also includes an adapter interface 110 at the proximal end 106 shaped and configured to align with and connect to an adapter linked to the Laser support system 100, such as the Laser support structure 160, during use.

Returning to FIG. 1, the processing system 164 is a computer system including a processing unit containing a processor and a memory. An output device, such as a display and input devices, such as keyboards, scanners, and others, are in communication with the processing unit. Additional peripheral devices also may be present. Data may be communicated to the processing system 164 by any known method, including by direct communication, by storing and physically delivering, such as using a removable disc, removable drive, or other removable storage device, over e-mail, or using other known transfer systems over a network, such as a LAN or WAN, including over the internet or otherwise. Any data received at the processing system may be stored in the memory for processing and manipulation by the processor. In some embodiments, the memory is a storage database separate from the processor. Other systems also are contemplated.

The processing system 164 may be configured and arranged to receive information over the wire 140, or through wireless communication methods that represent information or signals from the sensing devices 134. Using this information, the processing system 164 may be configured to calculate and output values or data representing the position of implant features, such as the interlock holes 108 of the nail implant 102, even when the implant 102 has been deformed if such sensory feedback is available and/or moved and is not visible to the surgeon. The system uses these features to identify access axes that allow a surgeon to access the implant in the patient in an effective manner. For example, a surgical guide such as a drill guide may be aligned with the interlock holes based on settings output from the processing system 164.

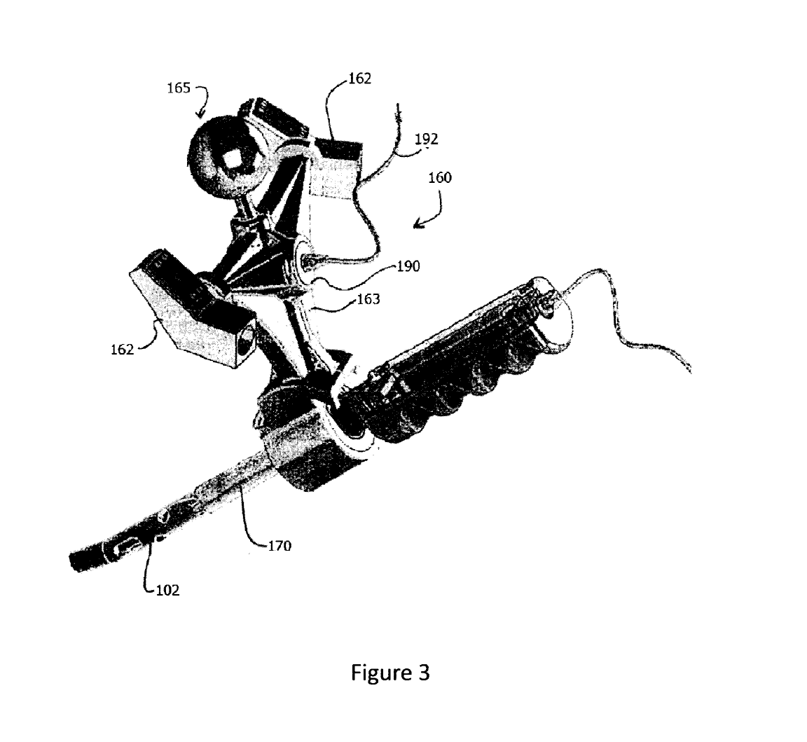

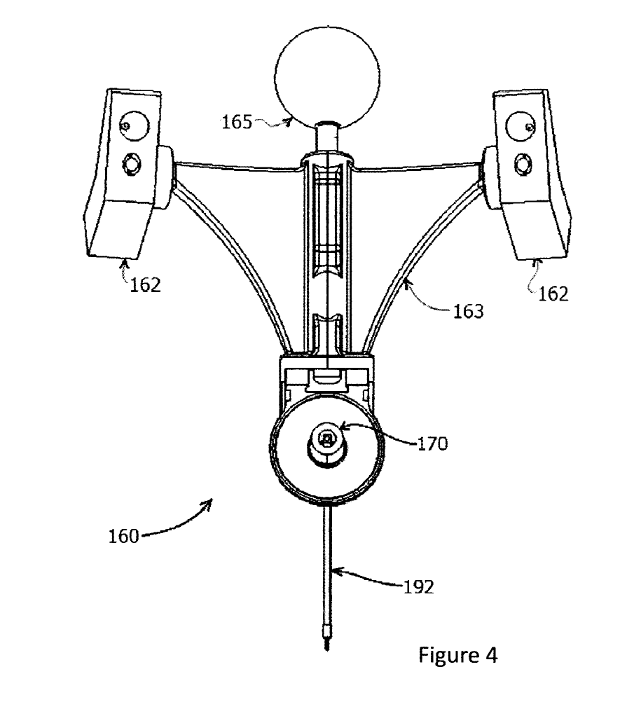

The Laser support structure 160 is shown in greater detail in FIG. 3-4. The Laser support structure 160 carries the Laser projectors 162. The Laser support structure 160 includes an implant adapter 170, a main body 163, and a multi-position adjustment mechanism 165. A main purpose of the Laser support structure 160 is to optimally orient the Laser projectors 162 such that the features to be targeted lie within a working conical envelope of the projection system 100.

FIG. 3 also shows a proximal end of the implant 102. The implant adapter 170 connects to the implant 102 with the use of a guide bolt that may thread into the implant 102.

The main body 163 is a rigid structure configured to maintain the two Laser projectors 162 in known position relative to the implant adapter 170 and the implant 102. In some embodiments, the implant adapter 170 includes an implant feature detector that may be, for example, a deflection probe (not shown) that interfaces with the implant, before, during, or after implantation that may be used to determine whether a feature of the implant (such as the interlocking screw holes in an IMN) has been deflected from an expected or original location. The deflection probe is configured to sense or otherwise determine whether the implant has deflected. Some of the deflection may be detected using strain measurements, such as strain gauges on the probe or on the implant. The processing system 164 may use this detected information to calculate the location of implant features that cannot be visually tracked (such as when an end of an IMN is implanted into bone, and potentially deflected by the bone) taking into account the strain. The Laser projection system can then determine where features of the implant are located so that it knows where the target axis for an implant or instrument should be generated. Here, the main body 163 includes an electrical connector outlet 190 configured to connect to the processing the system 164 through a connection element, such as a wire or cable 192. It should be noted that wireless systems are also contemplated, thereby reducing clutter in an operating room. Wired passages pass through the main body 163 providing electrical connection to the Laser projectors 162.

The Laser projectors 162 are carried by the main body 163 of the Laser support structure 160. Here, the Laser projection system 100 includes two Laser projectors 162 offset an equal distance from a centerline 161 through the Laser support structure 160, as can be seen in FIG. 4. It is worth noting that for some applications only one Laser projector 162 may be sufficient and still others may benefit from the use of more than two.

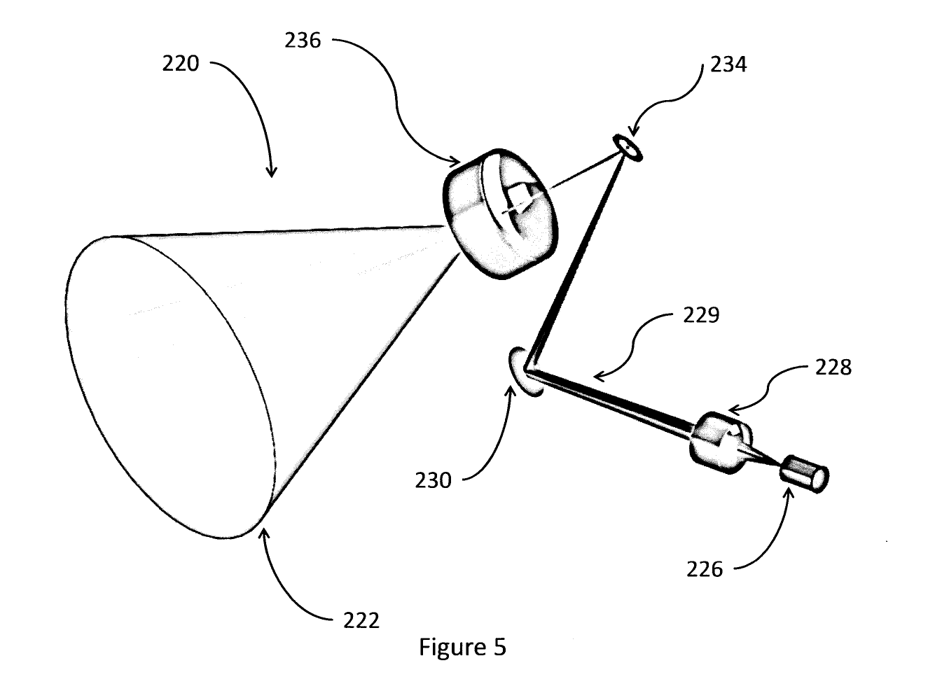

The Laser projectors 162 include an optical system 220 disposed therein. This optical system is shown in FIG. 5. A main objective of the optical system 220 is to provide a beam of light that originates from a given point in space that can be commanded to point at an arbitrary point in space. A working envelope 222 identifying the area or region within which a beam can be directed from the Laser projectors 162 is shown in FIG. 5. Although it can take many forms, here it is conical in nature. It could also be pyramidal or some other polygonal form.

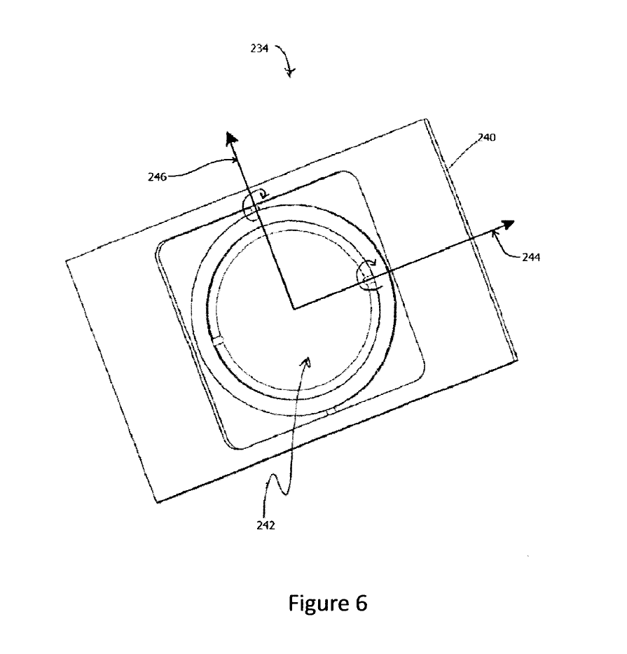

The optical system includes a Laser source 226, a collimator 228, a folding minor 230, a photo diode array 232, a MEMs minor 234, and an expansion lens 236. In this embodiment the Laser source 226 is a Laser diode. Typically, these generate an elliptical conical beam 221 which is passed through the collimator 228 to create a straight beam 229. This beam is then focused on the folding mirror 230. The folding mirror 230 is provided to, among other things, make the optical system 220 more compact. However, this isn't a requirement of the system 220 and will depend on the particular packaging requirements. The folding mirror 230 directs the beam to a micro-electro-mechanical system (MEMS) two-axis gimbal-less mirror 234 which bounces the light beam off in a desired elevation and rotation relative to the nominal. The MEMS mirror 234 is shown in FIG. 6. As can be seen, the MEMS minor 234 includes a base frame 240 and a mirror portion 242. The MEMs mirror portion 242 is rotatable about a first axis 244 and a second axis 246. Because some devices such as the MEMS mirror portion 242 have a limited angulation capability, the expansion lens 236 is used as shown in FIG. 5. In this embodiment, the expansion lens 236 expands the working envelope from roughly +/−2 degrees to +/−22 degrees, although other ranges are also contemplated. Although this embodiment utilizes MEMS technology, other more traditional means are available to manipulate a mirror in two-degrees of freedom such a motors, piezoelectric elements etc. Also other light sources other than Lasers are envisioned along with alternative means of collimating a light source.

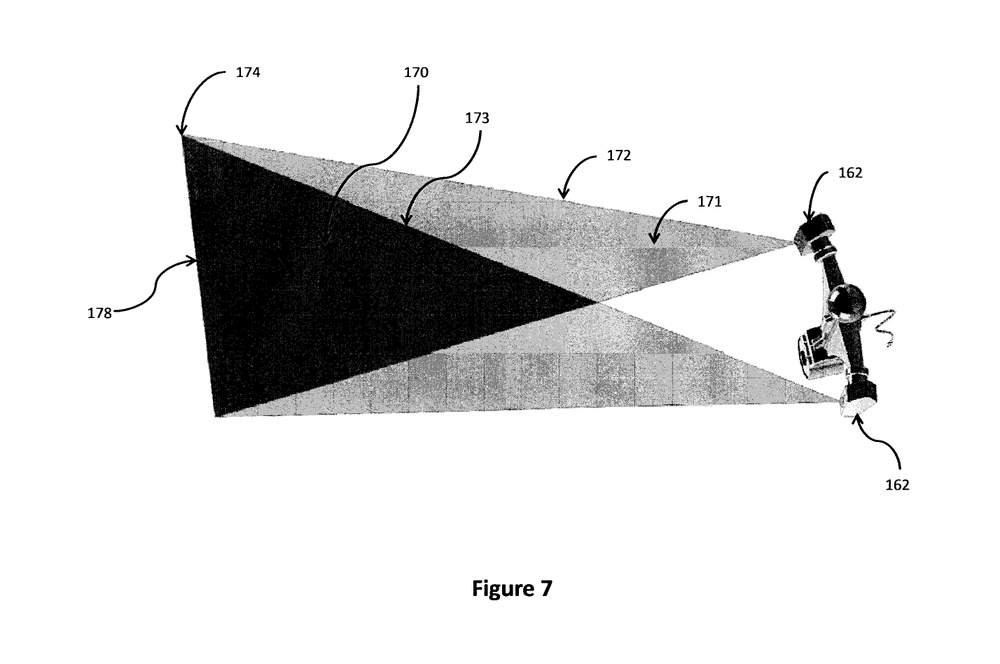

Referring to FIG. 7, each Laser projector 162 can target a specific point 163. If they are both targeting the same point in 3D space, the Laser beams from each projector 162 will cross at the point in space. Each Laser projector 162 can then be redirected to target a second point 165, sweeping along an angle between the two points. If each Laser projector cycles between these two points, light will illuminate a section of a plane 170, 171. If the two light sources are not coincident, then two plane sections can be illuminated such that the intersection of the two planes is the axis of interest 178.

Each Laser beam will pass through the air and illuminate the objects in their path. Typically, the light will strike the patient or surgical drapes. The user will place the instrument or implant, in this embodiment the drill guide 166 (shown in FIG. 1), in the area that is illuminated. Each light source will project a curve on the drill guide. If the drill guide shown in FIG. 1 includes a planar surface 168 perpendicular to the drill axis, then when the drill axis is aligned with the axis of interest 178 generated by the Laser system, each Laser projector 162 will illuminate a line on the planar surface 168 and these two lines will cross at the drill axis.

Theoretically, each Laser projector 162 defines an infinite plane. Practically, as shown in FIG. 7, each Laser projector 162 can illuminate only a sector of a plane 170, 171 within the working envelope of the Laser projector. By selecting the same point to define one edge 172, 173 of each illuminated plane sector 170, 171, the illuminated axis of interest includes on it a point of interest 174. This point of interest can be aligned with a feature of the implant or instrument. For example, a drill could be inserted in the drill guide 166 along the aligned with the illuminated axis of interest 178 until a mark is aligned with the point of interest 174, indicating that the target depth of the drill has been reached.

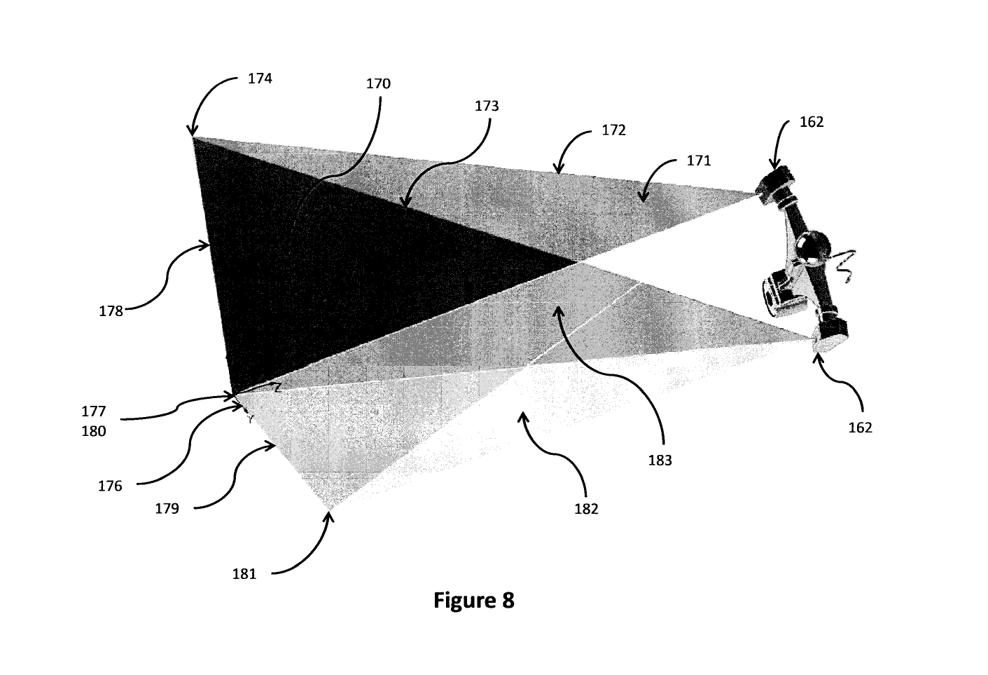

Further as shown in FIG. 8, a third point 180 and fourth point 181 could be selected such that each Laser projector 162 sweeps a section of a plane 182, 183 from the third point 180 to the fourth point 181. Because the Laser source 162 can be turned on and off at very high speed, only the desired plane sections will be illuminated by the Laser, and no extraneous light will be emitted while moving from projecting the first and second plane sections 170, 171 to the third and fourth plane sections. The intersection of the third and fourth plane sections is a second axis of interest 179. One use of this second axis 179 is to illuminate an axis that is coplanar but at an angle to the first axis 178, where the intersection of the first and second axis 178, 179 defines an origin 177 of a coordinate system 176 and the primary axes are aligned with the first and second axes of interest. This allows the surgeon to align an instrument or implant with the two axes of interest so that instrument or implant is placed in a desired location and pose.

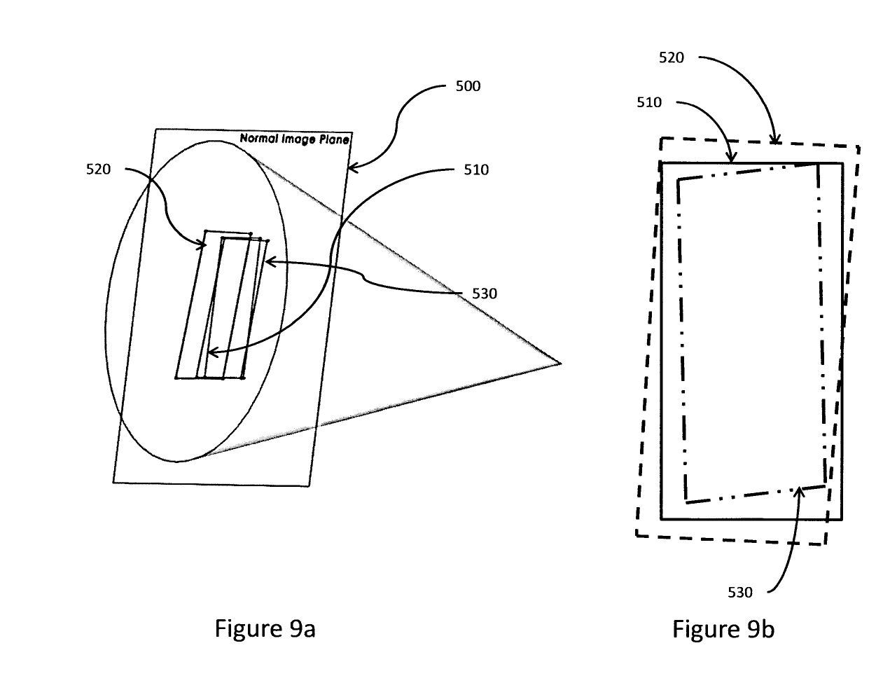

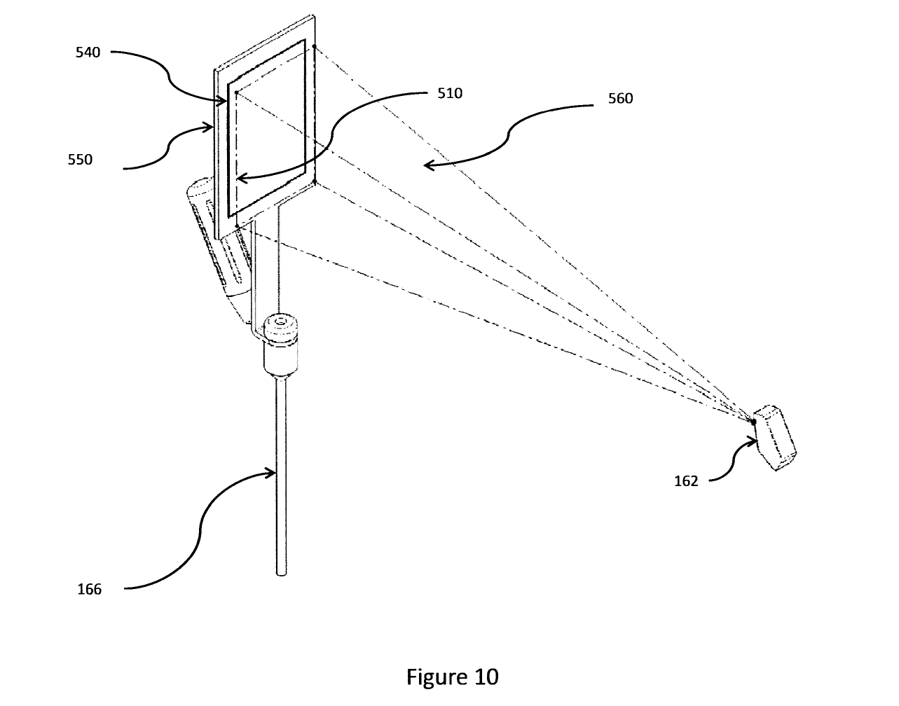

When it is desired to utilize a single light source to aid in the placement of an implant or instrument one may command that light source using the processing system, for example, to project a known shape as a location indicator onto a desired surface in 3d space that corresponds to a desired position of an implant or instrument in 3d space. A simple example of this would be projection of a rectangle as shown in FIGS. 9A and 9B where the desired surface is a plane 500 that has projected onto it the desired rectangle 510. When an implant or instrument has an inscribed rectangle of the same size and that implant or instrument is placed into the field of view of the light source then the light source will project onto it. If the implant or instrument is not perfectly aligned and positioned on the desired surface that is a plane 500, the form that is projected upon the implant or instrument will be skewed from the desired rectangular form 510 as is depicted in 520 and 530 in FIGS. 9A and 9B. The implant or instrument could then be repositioned, whether tilted, rotated, translated, such that the inscribed rectangular form present on the implant or instrument coincides with the projected form of the light source. FIG. 10 shows an instrument, in this case a drill guide 166 with a target 550 mounted to it with a rectangular form 540. The single Laser source 162 projects four sheets of light that when cast on a plane generate a polygon as a location indicator. When the target 550 is aligned with the sheets of light 560 such that the desired rectangle 510 aligns with the rectangular form 540, then the target is aligned with the desired plane 500 in the desired orientation.

In some situations forms other than rectangles might be better suited for the application in question but in each case the principle is the same, the light source projects a form onto a virtual desired plane, the implant or instrument has inscribed onto it a similar form and that implant or instrument is positioned within the field of view of the light source until the projected and inscribed forms coincide. That is, when the projected and inscribed forms coincide, then the implant or instrument is aligned or posed as desired. While described as a location indicator with a rectangular shape, the location indicator and any inscribed surface or target could be any shape and could be an open shape, such a series of lines or as an X shape, or may be closed-shaped, such as the rectangle enclosing an area.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A Laser projection system for identifying a point in space; comprising:
a first Laser projector configured to generate a first beam at a first angle;
a second Laser projector configured to generate a second beam that intersects the first beam at an intersection; and
a system configured to determine a location of a point of the intersection of the first and second beams in three-dimensional space.

2. The Laser projection system of claim 1, wherein the first and second Laser projectors are each configured to generate beams as planar sheets, the intersection of the sheets forming an axis, the system being configured to determine a location of the axis.

3. The Laser projection system of claim 2, further comprising a surgical drilling instrument, and wherein the axis is aligned with an outer surface of the drilling instrument for drilling to the feature of an implant in a patient.

4. A Laser projection system for marking an axial target as a part of a surgical implant procedure; comprising:
a first Laser projector configured to project a first plane of light; and
a second Laser projector configured to project a second plane of light that intersects the first plane of light; and
a system configured to manipulate the Laser projectors so that a line defined by the intersection is aligned to represent the axial target as one of a location of a feature of an implant implanted within a patient and a desired location of an instrument used for implantation of an implant.

5. The Laser projection system of claim 4, wherein the line is aligned with an outer surface of an instrument for drilling to the feature of the implant.

6. The Laser projection system of claim 4, wherein each of the first and second Laser projectors comprise a minor that is vibrated to create the respective first and second planes of light.

7. A Laser projection system for marking a positional target as part of a surgical implant or instrument, comprising:
a Laser projector configured to project a visual indicator onto a virtual surface in space at a location on the virtual surface coinciding with the desired placement of a surgical implant or instrument; and
a surgical implant or instrument comprising an outer surface such that when the surgical implant or instrument is placed in a field of view of the Laser projector, the projected visual indicator aligns on the outer surface when the implant or instrument is properly placed in space.

8. The Laser projection system of claim 7, wherein the visual indicator is a line.

9. The Laser projection system of claim 7, further comprising a second Laser projector configured to project a second visual indicator intersecting the first visual indicator to form an intersection representing an axis coinciding with the desired placement of a surgical implant or instrument.

10. The Laser projection system of claim 9, wherein the Laser projectors are configured to project the indicators on patient anatomy to identify a point coincident with an axis of interest.

11. A Laser projection system for marking an axial target as a part of a surgical implant procedure performed by a surgeon, comprising:
an implant feature locator configured to determine the location of a feature of an implant in a patient and determine a target axis to the feature of the implant;
a first Laser generating system configured to generate a Laser sheet passing through the target axis for the feature of the implant; and
a second Laser generating system offset from the first Laser generating system configured to generate a Laser sheet passing through the target axis for the feature of the implant, the second Laser sheet intersecting the first Laser sheet at the target axis in a manner visible to the surgeon.

9. The Laser projection system of claim 11, wherein the implant feature locator comprises a processing system configured to receive and process dimensional data of the implant to determine the location of the feature of the implant.

10. The Laser projection system of claim 12, wherein the processing system is configured to receive data from a deflection probe associated with the implant and configured to calculate deformation of the implant and determine the desired location of the target axis.

11. The Laser projection system of claim 11, wherein the target axis is an axis configured to align with an outer surface of an instrument for drilling to the feature of the implant.

12. The Laser projection system of claim 11, wherein each of the first and second Laser generating systems comprises a mirror that is vibrated to create the Laser sheet.

13. The Laser projection system of claim 11, wherein the first Laser generating system comprises a cylindrical lens that is rotated to create the Laser sheet.

14. The Laser projection system of claim 11, wherein the first Laser generating system comprises a minor disposed on a MEMS chip that moves the mirror in two planes to direct a Laser and form the first Laser sheet.

15. The Laser projection system of claim 11, wherein the implant is one of an intramedullary nail, a hip replacement, and a knee replacement.

16. A method for marking an axial target as a part of a surgical implant procedure, comprising:
implanting an implant in a patient, the implant comprising a feature of interest; and
using a first Laser beam and a second Laser beam that intersect to create a visual point on patient's anatomy identifying a target to the feature of interest of the implant.

17. The method of claim 16, comprising generating the first Laser sheet and a second Laser sheet that intersect create a visual intersecting axis, the visual point being along the intersecting axis, the intersecting axis identifying a target axis to the feature of interest of the implant.

18. The method of claim 16, comprising maintaining a surgical instrument in a position where the visual intersecting axis forms a line on the instrument to provide an indicator of whether the surgical instrument is aligned with the target axis to the feature of interest of the implant.

19. A method of determining the location of a surgical implant or instrument, the method including:
implanting a reference implant;
determining the location of the reference implant relative to a Laser system;
illuminating one or more points identifying a location relative to the reference implant with the Laser system; and
aligning instruments or implants utilizing the one or more illuminated points.

20. The method of claim 19, wherein the Laser system comprises two spatially separate Laser sources and wherein illuminating one or more points with the Laser system comprises orienting Lasers from the two Laser sources so that they intersect to illuminate the one or more points with the Laser system.

21. The method of claim 20, wherein illumination of one or more points comprises:
illuminating items placed in a section of a first plane generated by the first Laser source;
illuminating items placed in a section of a second plane generated by the second Laser source, wherein the intersection of the first plane and the second plane defines an axis of interest; and
aligning instruments or implants comprises utilizing the axis of interest.

22. The method of claim 21, comprising:
illuminating items placed in a section of a third plane generated by the first Laser source; and
aligning the implant or instrument using the third plane.

23. The method of claim 22, comprising:
illuminating items placed in a section of a fourth plane generated by the second Laser source, wherein the intersection of the third plane and the fourth plane defines a second axis of interest; and
aligning instruments or implants comprises utilizing the first axis of interest and second axis of interest.

24. The method of claim 21, wherein the section of the first plane and the section of the second plane end at a desired point of interest along the axis of interest, and
wherein aligning instruments or implants comprises aligning along the axis of interest to a position relative to the point of interest.

25. The method of claim 19, comprising projecting the one or more illuminated points with a single Laser.
